(19) United States
(12) Patent Application Publication
(10) Pub. No.: US 2008/0146823 A1
(43) Pub. Date: Jun. 19, 2008

(54) METHOD OF MAKING PURIFIED ODOR FREE TOCOPHEROL ACETATE

(76) Inventors: Madhu V. Kulkarni, Bourbonnais, IL (US); George R. Martin, Bourbonnais, IL (US)

Correspondence Address: COGNIS CORPORATION;PATENT DEPARTMENT, 300 BROOKSIDE AVENUE, AMBLER, PA, 19002, US

(21) Appl. No.: 12/034,001

(22) Filed: Feb. 20, 2008

Related U.S. Application Data
(63) Continuation of application No. 11/451,627, filed on Jun. 12, 2006, now abandoned.
Application No. 11/451,627 is a continuation of application No. 11/018,842, filed on Dec. 21, 2004, now abandoned.
Application No. 11/018,842 is a continuation of application No. 10/371,393, filed on Feb. 21, 2003, now abandoned.
Application No. 10/371,393 is a continuation of application No. 10/146,161, filed on May 15, 2002, now abandoned.
Application No. 10/146,161 is a continuation of application No. 09/801,500, filed on Mar. 8, 2001, now abandoned.
Application No. 09/801,500 is a continuation of application No. 09/552,254, filed on Apr. 19, 2000, now abandoned.
Application No. 09/552,254 is a continuation of application No. 08/656,592, filed on May 31, 1996, now abandoned.
Application No. 08/656,592 is a continuation of application No. 08/639,943, filed on Apr. 18, 1996, now abandoned.
Application No. 08/639,943 is a continuation of application No. 08/380,463, filed on Jan. 27, 1995, now abandoned.

(51) Int. Cl. C07D 311/72

(52) U.S. Cl. 549/410

(57) ABSTRACT

A process for producing odor free tocopherol acetate is disclosed. This process comprises forming a solution of tocopherol acetate in a suitable solvent, e.g., isopropyl alcohol, subjecting said solution to a sufficient amount of low pressure and heat to remove said solvent and sparging sufficient amount of an inert gas through said solution to remove traces of remaining solvent.

### FIELD OF THE INVENTION

The present invention relates to a method and more particularly, it relates to a method of preparing highly purified odor free alpha-tocopherol acetate having the following structural formula (I).

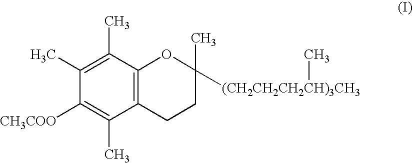

### BACKGROUND OF THE INVENTION

Vitamin E designates a group of naturally occurring compounds known as tocopherols which comprise, for example, alpha-tocopherol, beta-tocopherol, gamma-tocopherol and delta-tocopherol. These compounds are found, for example, in vegetation, seeds, cereals, nuts and the like.

Vitamin E is known as an membranous antioxidant. It plays a part in the membranous parts of cells. Thus, it interdigitates with phospholipids, cholesterol and triglycerides, the three main structural elements of membranes. Because Vitamin E acts as an antioxidant, it reacts at the cell sites with the destructive compounds known as free radicals. Vitamin E converts these free radicals into a less harmful form. In addition, Vitamin E is said to have an antisterility function. Therapeutically, it is indicated for a variety of clinical conditions, such as prevention of abortion, improve fertility, in addition to its nutritional values administered in the form of dietary supplements. Of the tocopherols, d-alpha-tocopherols has the greatest biological activity.

### DESCRIPTION OF PRIOR ART

The tocopherols including the important alpha-tocopherols are fat soluble. In some humans and animals they are not well absorbed. Consequently, alternate tocopherol derivatives have been proposed. See, for example, U.S. Pat. No. 2,680,749. These include the tocopherol acetate (I) which is described above.

Briefly, Compound I can be prepared by treating alpha-tocopherol with acetic anhydride in the presence of sodium acetate. The resulting compound is further purified by molecular distillation followed by crystallization in isopropanol solution. However, the compound thus obtained may not be totally odor free or sufficiently pure for therapeutic purposes. Obviously, it is desirable to obtain a higher purity product which is odor free.

### SUMMARY OF THE INVENTION

A novel method of making highly purified and odor free compound (I) has been found. This method comprises first forming the acetate by reacting the tocopherols with acetic anhydride in the presence of sodium acetate followed by purification steps of molecular distillation and low temperature crystallization in a suitable solvent in that order. A solution of tocopherol acetate in isopropanol is preferred. The solvent is removed by stripping under reduced pressure. When the bulk of solvent has been removed, an inert gas such as nitrogen is sparged into the acetate to remove the last traces of solvent and odor yielding odor free highly purified tocopherol acetate suitable for therapeutic purposes. It is also possible to use steam as a stripping media, steam can be made in situ by directly adding water to the stripping kettle immediately after stripping the solvent.

### DESCRIPTION OF THE INVENTION

The present invention relates to a method and more particularly, it relates to a method of making highly purified odor free alpha-tocopherol acetate having the structural formula (I) as described above.

According to the present invention, therapeutic grade tocopherol acetate is produced by first reacting tocopherol with acetic anhydride and sodium acetate. The resulting product is then washed with water to remove excess of acetic anhydride and sodium acetate. It is purified by molecular distillation followed by low temperature crystallization in a suitable solvent, such as lower molecular weight alcohols. Isopropanol is the preferred solvent, however, other volatile solvents can also be used. The crystallized tocopherol acetate is separated from the bulk of the solvent by filtration. The remaining solvent from the crystallized acetate is removed by gradually heating the product under a low pressure of about 30 to 25 mm of hg, typically 25 mm. The temperature is controlled at about below 90° C. When the pressure and temperature have reached the optimum level the solution is treated with a stream of an inert gas, such as nitrogen or steam. Thus for example, nitrogen is introduced by sparging. The treatment under nitrogen continues until the last traces of the solvent has been removed. The material is submitted to lab for odor test. The solvent free product is allowed to cool to room temperature while gradually breaking the vacuum using nitrogen. The product is then filtered using a suitable filter having a pore size of about 1 micron.

The present process is also suitable for treating tocopherol acetate as it is prepared from batch process having a content of about 25% by weight to about 50% by weight in isopropyl alcohol. It is equally suitable for purifying tocopherol acetate of commerce. Thus, a solution of tocopherol acetate is prepared by dissolving it in isopropyl alcohol and then treated as described above.

In order to further illustrate the practice of the present invention, the following Example is included.

### EXAMPLE

A 50% by weight solution of tocopherol acetate in isopropyl alcohol, is charged to a kettle equipped with a heating and vacuum system. With agitation, the kettle, is slowly heated until a temperature of about 90° C. is attained. Care is taken not to exceed this temperature. A vacuum is applied until about 25 mm of pressure has been reached. When the desired pressure and temperature has been attained, it is held at this temperature and pressure. Nitrogen at room temperature is sparged through the liquid until last traces of isopropyl alcohol have been removed.

The solution is then allowed to cool to room temperature and the vacuum is broken by introducing gaseous nitrogen. The cooled product is filtered using a filter having an approximate pore size of about 1 micron yielding a highly purified odor free tocopherol acetate.

What is claimed is:

1. A process for the production of highly purified odor free tocopherol acetate which comprises:
a. forming a solution of tocopherol acetate in a volatile solvent;
b. subjecting said solution to a sufficient amount of low pressure and heat to remove said solvent; and
c. sparging a sufficient amount of an inert gas through said solution to remove traces of remaining solvent.

2. A process according to claim 1 wherein said solvent is isopropyl alcohol.

3. A process according to claim 2 wherein about a 25% by weight to about a 50% by weight of tocopherol acetate is formed in isopropyl alcohol.

4. A process according to claim 1 wherein a low pressure of about 25 mm to about 30 mm and a temperature of about below 90° C. are applied to said solution in step “b”.

5. A process according to claim 1 which further comprises the step of filtering the product obtained in step “c”.

6. A process according to claim 5 wherein said filtering is effected with a filter having a pore size of about 1 micron.

7. A process according to claim 3 wherein about a 50% by weight of tocopherol acetate is formed in isopropyl alcohol.

8. A process according to claim 4 wherein about 25 mm of pressure is applied to step “b”.

9. A process according to claim 1 wherein said inert gas is nitrogen.

10. A process according to claim 1 wherein said inert gas is steam.
